(19) United States
(12) Patent Application Publication
(10) Pub. No.: US 2009/0281536 A1
(43) Pub. Date: Nov. 12, 2009

(54) Medical Device For Diagnosing and Treating Anomalous Tissue and Method for Doing the Same

(76) Inventors: Hugh Beckman, Boca Raton, FL (US); Terry A. Fuller, Jenkintown, PA (US); Richard L. Beckman, New York, NY (US)

Correspondence Address: YOUNG BASILE, 3001 WEST BIG BEAVER ROAD, SUITE 624, TROY, MI, 48084, US

(21) Appl. No.: 12/437,589

(22) Filed: May 8, 2009

Related U.S. Application Data
(60) Provisional application No. 61/051,705, filed on May 9, 2008.

(51) Int. Cl. A61B 18/18; A61B 6/00; A61B 18/16; A61N 7/00

(52) U.S. Cl. 606/33; 600/476; 606/41; 601/2

(57) ABSTRACT

Disclosed herein are medical devices for diagnosing and treating anomalous tissue and methods of use. One embodiment of the medical device can comprise an energy source configured to emit at least an excitation beam and a therapeutic beam, a probe coupled to the energy source and configured to propagate the excitation and therapeutic beams with the beams capable of contact with the tissue, a sensor coupled to the probe that detects at least one predefined attribute of radiation emanating from the tissue when the tissue is subjected to the excitation beam and a controller coupled to the energy source and the sensor and programmed to selectively alternatively actuate the energy source to emit the excitation beam and the therapeutic beam in response to the detection of the at least one predefined attribute by the sensor.

### CROSS-REFERENCE TO RELATED APPLICATION

This application claims priority from U.S. Provisional Application Ser. No. 61/051,705, filed May 9, 2008, which is incorporated herein in its entirety by reference.

### FIELD OF THE INVENTION

The present invention relates in general to devices for diagnosing tissue via detecting spectra and the linking of those devices to a therapeutic modality for the concurrent diagnosis and treatment of abnormal tissue.

### BACKGROUND

Surgical excision of neoplastic tumor tissue has historically been performed manually using steel blades and lasers. In recent years, robotic devices have been employed to assist the surgeon. Currently, many surgeons advocate the use of the Mohs technique to diagnose and remove malignant tissues. The Mohs technique includes taking a mapped specimen of tumor tissue, staining the tissue, and evaluating the tissue under a microscope to determine the amount and location of the residual tumor cells. In particular, the area with the tumor is marked and frozen with a local anesthetic. The tissue is surgically removed, divided and mapped with reference points on the patient. The slides of the frozen sections are analyzed by the surgeon. If any section of the slide contains tumor, the map guides the surgeon to the precise location where the tumor root remains. This process is repeated until no tumor is seen on the slides. There are many disadvantages to this treatment system. There may be unnecessary tissue removal and cosmetic damage. Lengthy treatment sessions are necessary due to the manual viewing and determination of cancer cells within each layer removed. Freezing of tissue samples may also be required, which can affect the accuracy of the analysis. A simpler, more efficient and concurrent method of diagnosis and removal of abnormal tissue would represent a significant enhancement for patient care.

### BRIEF SUMMARY

Disclosed herein are embodiments of a medical device for diagnosing and treating anomalous tissue. Selected embodiments are summarized here. In one embodiment the medical device comprises an energy source configured to emit at least an excitation beam and a therapeutic beam, a probe coupled to the energy source and configured to propagate the excitation and therapeutic beams with the beams capable of contact with the tissue, a sensor coupled to the probe that detects at least one predefined attribute of radiation emanating from the tissue when the tissue is subjected to the excitation beam and a controller coupled to the energy source and the sensor and programmed to selectively alternatively actuate the energy source to emit the excitation beam and the therapeutic beam in response to the detection of the at least one predefined attribute by the sensor.

In another embodiment, the medical device comprises a probe, a spectrometer coupled to the probe, a database of tissue fingerprints, a controller coupled to the probe and the spectrometer and a first energy source coupled to the probe and configured to emit one or more of a diagnostic excitation, a therapeutic ablation and a diagnostic ablation as directed by the controller or a user on a target tissue. The first energy source delivers excitation energy through the probe to the tissue during the diagnostic excitation and the spectrometer receives a scatter from the diagnostic excitation and identifies the scatter against the database, the controller receiving a signal from the spectrometer of normal or abnormal. The first energy source can also deliver ablative energy through the probe to an anomalous target tissue during the therapeutic ablation depending on the signal and deliver ablative energy through the probe to normal target tissue during the diagnostic ablation depending on the signal.

In yet another embodiment, the medical device for diagnosing and treating anomalous tissue comprises an electromagnetic energy source, a Raman spectrometer, a central processing unit having a database of tissue fingerprints and a probe configured to deliver the electromagnetic energy to perform a diagnostic excitation, a therapeutic ablation and a diagnostic ablation in any order as directed by the controller or a user on a target tissue. The diagnostic excitation comprises delivering excitation electromagnetic energy from the electromagnetic energy source through the probe to the target tissue suitable to cause Raman scattering, collecting a Raman scatter produced by the target tissue with the probe and delivering the Raman scatter to the Raman spectrometer, fingerprinting the Raman scatter with the Raman spectrometer, comparing the fingerprint to the database of tissue fingerprints and determining if the target tissue is anomalous. The therapeutic ablation comprises delivering ablative electromagnetic energy through the probe from the electromagnetic energy source to an anomalous target tissue, and the diagnostic ablation comprises delivering ablative electromagnetic energy through the probe from the electromagnetic energy source to a normal target tissue. The therapeutic and diagnostic ablation source can be any combination of a coherent or incoherent electromagnetic energy source, electrosurgical generator or plasma scalpel.

Also disclosed are methods of diagnosing and treating anomalous tissue with the medical device. One such method comprises positioning a probe coupled to an energy source proximate a target tissue, delivering one of an excitation energy and an ablative energy through the probe from the energy source to the target tissue depending on a signal from a controller, capturing a scatter reflected from the target tissue with the probe when excitation energy has been delivered, relaying the scatter to a spectrometer and fingerprinting the scatter's spectra against a tissue fingerprint database in the controller and providing the signal from the controller to the energy source.

### BRIEF DESCRIPTION OF THE DRAWINGS

The description herein makes reference to the accompanying drawings wherein like reference numerals refer to like parts throughout the several views, and wherein:

FIG. 1 is a schematic of an embodiment of a medical device for diagnosing and treating anomalous tissue as disclosed herein;

FIG. 2 is a cross-sectional view of a probe used in embodiments of the medical device for diagnosing and treating anomalous tissue;

FIG. 3 is a flow diagram depicting the operation of embodiments of the medical device for diagnosing and treating anomalous tissue;

FIG. 4 is an example of normal tissue fingerprints;

FIG. 5 is an example of anomalous fingerprints;

FIG. 6 is a cross-sectional view of a second embodiment of a probe used in the medical device for diagnosing and treating anomalous tissue;

FIG. 7 is a cross-sectional view of a third embodiment of a probe used in the medical device for diagnosing and treating anomalous tissue;

FIG. 8 is a cross-sectional view of a fourth probe embodiment used in the medical device for diagnosing and treating anomalous tissue;

FIG. 9A is a schematic of a fifth embodiment of a probe used in the medical device for diagnosing and treating anomalous tissue; and

FIG. 9B is an exploded view of the fifth embodiment of a probe used in the medical device for diagnosing and treating anomalous tissue.

### DETAILED DESCRIPTION OF EMBODIMENTS OF THE INVENTION

In the various figures, like reference numbers refer to like parts. The figures are exemplary and are not drawn to scale.

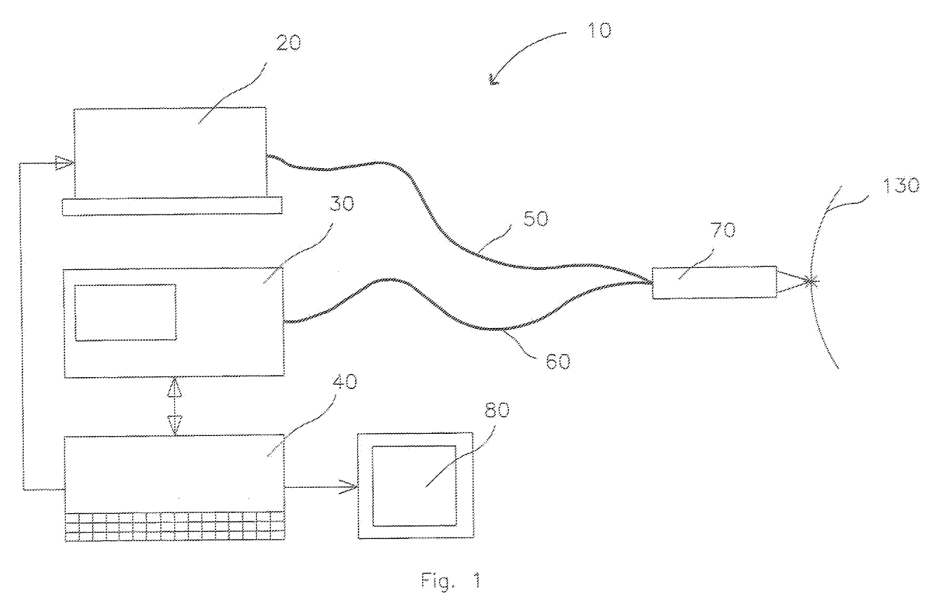

FIG. 1 illustrates one embodiment of a medical device for diagnosing and treating anomalous tissue disclosed herein. The medical device 10 comprises emitter or energy source 20, sensor 30, controller 40 and probe 70. Optional display 80 can be connected to controller 40. Probe 70 can comprise excitation/ablation conduit 50 and collection or sensing conduit 60. Probe 70 can be configured to enclose at least a portion of the conduits 50, 60 at their distal ends. Energy or light source 20, sensor 30, controller 40, probe 70 and display 80 can communicate with one another via communication links depicted by the arrows between the units. The communication links can transmit information, for example, through cables running from unit to unit. It is also contemplated that the communication links can be wireless, such as infra-red or radiofrequency. The communication links described are provided by way of example and not limitation, and other methods of communication can be used by those skilled in the art.

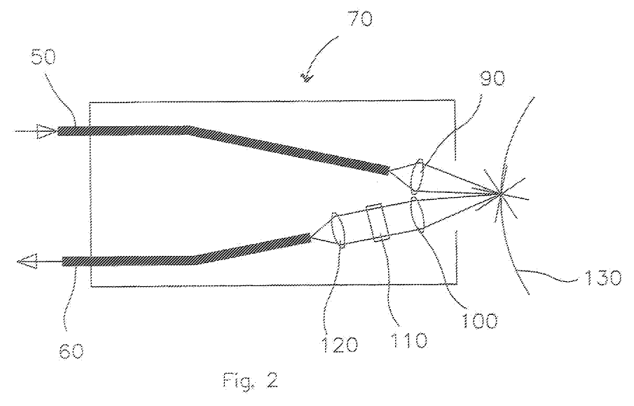

FIG. 2 is an exploded cross-sectional view of probe 70. In addition to comprising at least a portion of excitation/ablation conduit 50 and sensing conduit 60, this first probe embodiment can also comprise lens 90 in the path from the distal end of excitation/ablation conduit 50 and first lens 100, filter 110 and second lens 120 in the path from tissue 130 to the distal end of sensing conduit 60.

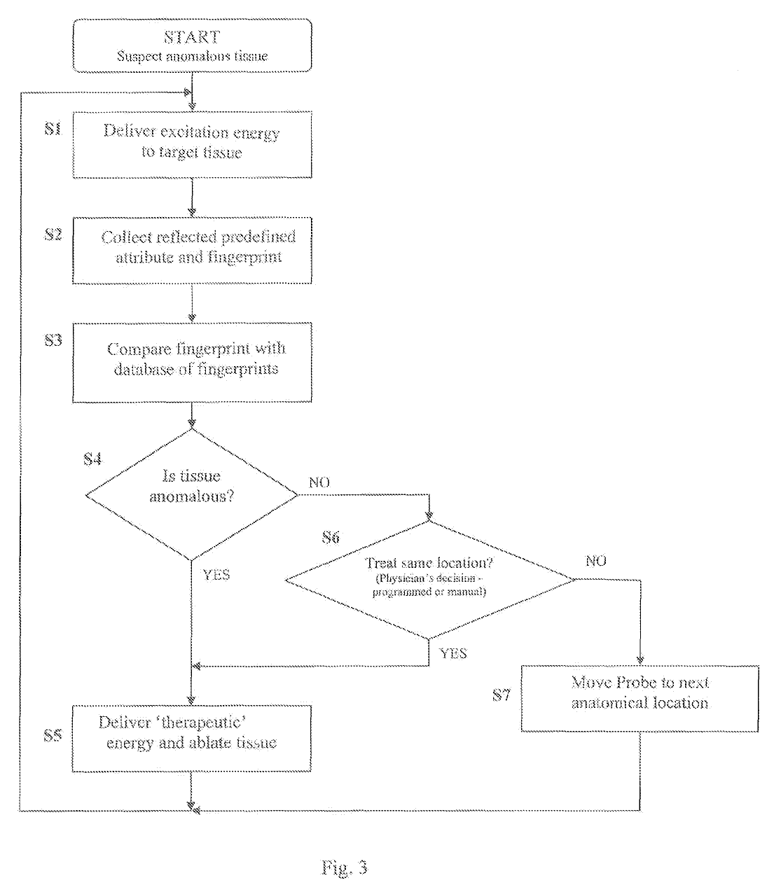

Medical device 10 with probe 70 can be used as follows. Probe 70 is positioned with its distal end relative to target tissue site 130. The positioning of probe 70 during a procedure can be, for example, within one to two millimeters of target tissue 130, or can contact target tissue 130. This distance is provided by way of example and not limitation, and any distance known to be practiced by those skilled in the art is contemplated. As used herein, the term “procedure” refers to any use of embodiments of the medical device on a patient. Referring to FIG. 3, the various uses of medical device 10 will be described.

Medical device 10 can be used to diagnose target tissue 130. The general tissue area targeted to undergo a procedure can be determined by any process known to those skilled in the art to assess tissue conditions. A non-limiting example can be a physician visually determining that a spot on skin is suspect and requires further assessment Another non-limiting example might be that the tissue is already known to be malignant, thus requiring removal. As used herein, “target tissue” is any tissue, whether normal, malignant, or denatured that is subject to diagnosis. Once probe 70 is positioned at target tissue 130, energy source 20 can be triggered to deliver an excitation beam through probe 70 to target tissue 130 (S1).

As used herein, the terms “energy” and “light” refers to ultraviolet, visible or infrared electromagnetic energy. However, it is to be understood that other appropriate forms of electromagnetic energy can be used by those skilled in the art. For example, a plasma scalpel as well as an electrosurgical device can be used with the medical device for therapeutic ablation.

The beam of diagnostic excitation energy can pass through lens 90 to focus the excitation energy if desired or required directly on target tissue 130 to be diagnosed. Probe 70 and/or conduit 50 can be, by way of example, a fiber optic made of quartz, sapphire, or other energy transmitting material with diameters in the range of about 100 μm to about 600 μm. Probe and/or conduit 50 can be any length required, and specifically can be two to four meters. Diagnostic excitation energy is that energy sufficient to deliver photons incident to target tissue 130 without damaging the tissue. Typical excitation wavelengths include 785 nm, 830 nm, 632.8 nm and 532 nm. Longer wavelengths, such as 1,064 nm, 980 nm and 810 nm can also be used, Depending on the focal spot size, the power to the tissue can be in the range of about 5 mw to about 500 mw. Focal beam diameters can be as small as 20 micrometers. The excitation power can vary depending upon the sensitivity of the spectrometer, the tissue spot size and the absorption characteristics of the tissue. Non-limiting examples of energy sources that can produce diagnostic excitation light with the appropriate strength include carbon dioxide, homium, Nd:YAG, diode, and argon. The use of lasers and other light sources known to those skilled in the art to produce the desired energy is also contemplated.

The photons of the diagnostic excitation beam incident on target tissue 130 produce at least on e predefined attribute of radiation, such as a scatter. The sensor 30 detects the at least one predefined attribute of radiation emanating from the tissue when the tissue is subjected to the excitation beam. By means of example and not limitation, embodiments herein will be described using Raman scatter as the at least one predefined attribute of radiation. The Raman scatter varies depending on the molecules found in target tissue 130. The incident photons can be collected by probe 70 and relayed to sensor 30 (S2). An example of a sensor for use with Raman scatter is a spectrometer. Probe 70 and/or collecting/sensing conduit 60 can be a fiber optic made of quartz (fused silica, or other optical materials well known in the art, similar to excitation/ablation conduit 50. Sensing conduit 60 is preferably a multimode fiber but can be a single mode fiber or fiber bundle. Its length should generally match that of conduit 50. As seen in FIG. 2, the excitation/ablation optical path and the collection path are at angles to target tissue 130.

Spontaneous Raman scattering is typically very weak, and as a result, the weak scattered light should be separated from the intense Rayleigh scattered light. Rayleigh scattering is defined as the scattered light that is the same energy as the incident excitation light. To address this, the Raman scatter can be collected by first lens 100 and passes through filter 110 and second lens 120. First lens 100 directs the Raman scatter to filter 110, where the Rayleigh scatter is removed. Non-limiting examples of filters that can be used include long pass filters, edge filters, band pass filters, notch filters and diffraction gratings. The Raman scatter passing through filter 110 can be focused through second lens 120 to be collected by sensing light conduit 60. Sensing light conduit 60 carries the Raman scatter to spectrometer 30. Any suitable spectrometer can be used. A non-limiting example of a suitable spectrometer is the Perkin-Elmer RamanFlex 400 Fiber Optic Raman analyzer. It should be noted that the location of the filter 110 is not limited to that shown in FIG. 2. Many spectrometers known in the art incorporate a filter of the type used here into the hardware of the spectrometer. It is contemplated that this type of spectrometer can eliminate the need for filter 110 as shown here. Other predefined attributes of radiation may not be as weak as the Raman scatter, reducing or eliminating the need for some or all of lenses and filters. The combination of lenses and filters can be adapted as desired or required to best produce and collect the attribute.

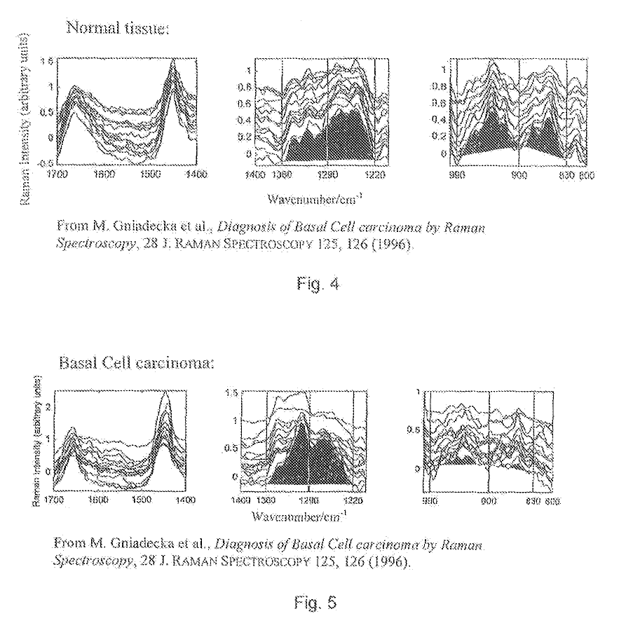

Spectrometer 30 receives the Raman scatter and can fingerprint the scatter. As used herein, “fingerprint” refers to the wavelength and intensity of the spectral distribution produced by spectrometer 30 that are associated with the scatter produced by the incident photons. The composition of target tissue 130 can be determined from the wavelength and intensity of the spectral distribution, or the fingerprint, produced by spectrometer 30. FIGS. 4 and 5 are examples of fingerprints from a spectrometer. FIG. 4 is Raman spectra of healthy skin. FIG. 5 is Raman spectra of basal cell carcinoma.

The resultant fingerprint can be used to determine the composition of target tissue 130 (S3). This determination can be made by comparing the fingerprint to a memory or database of known fingerprints. The database, which can be populated by multivariate analyses of samples of tissue anomalies, normal tissue samples, and varying degrees of denatured tissue samples. As used herein, “denatured tissue samples” refer to either normal or malignant tissue that has been thermally denatured by varying degrees of ablation or have any degree of overlying char due to ablation, as denatured tissue will have a different pattern of scatter.

It is also contemplated that the actual fingerprint of the anomaly can be obtained during a biopsy and programmed into the computer for comparison during treatment with medical device 10. It is also contemplated that normal tissue fingerprint data can populate the database, and an anomalous fingerprint can be determined by variations from the normal tissue fingerprints. It is further contemplated that fingerprint data of denatured tissue be stored in the database and an anomalous fingerprint can be determined by comparison with the denatured fingerprint data as well. The memory or database can be configured to store each fingerprint. The fingerprints can be associated with the particular treatment session and/or added to the database for future use as desired or required. Display 80 can be configured to display the fingerprints as a virtual biopsy for documentation of anomalous tissue removed. It is also contemplated that the surgeon can view the fingerprint or virtual biopsy displayed and make the determination of normal or anomalous. The functions of the database are provided by way of example and not limitation, and other uses of the database well known in the surgical art are contemplated.

Controller 40, or surgeon where desired, determines based on the fingerprint of target tissue 130 whether target tissue 130 is anomalous or normal (S4). As used herein, the term “anomalous” or “anomaly” refers to that tissue which is desirable to remove. The anomaly can be, for example, cancer or precancerous lesions or abnormalities and other pathology. If the determination is made that the fingerprint of target tissue 130 is anomalous, then controller 40 actuates the energy source 20 to emit a therapeutic beam, initiating ablation of the target tissue 130 (S5). As used herein, an “anomalous fingerprint” can be a malignant fingerprint not yet ablated and a denatured anomalous fingerprint that has been one or more times ablated but not yet free of malignancy. Energy source 20, through probe 70, delivers ablative energy to that same target tissue 130 sufficient to ablate at least a portion of target tissue 130. This therapeutic ablative light delivered by energy source 20 may be delivered in one or a plurality of doses as desired or required, or in a continuous mode. One or both of the intensity of the dose and the duration of the dose may be varied as required to sufficiently ablate the anomalous tissue. As used herein, the term “ablate” refers to effectively removing the anomaly by separation or destruction by vaporization, evaporation, melting, or the like. Non-limiting examples of sources that can produce the therapeutic ablative with the appropriate strength are provided in Table 1. Energy source 20 can be used to deliver both the diagnostic excitation beam and the therapeutic beam as described, using appropriate wavelengths and irradiance depending on the trigger or signal received from the controller 40 or surgeon. It is also contemplated that separate energy sources can be used, one producing the diagnostic beam and another producing the therapeutic, or ablative, beam. Energy sources are not limited to light sources such as lasers and can be any light source known to those skilled in the art that is sufficient to achieve the results desired. One or more lenses can be used to focus the therapeutic laser energy on the target tissue to be ablated.

TABLE 1

| | | | Typical Max | Typical |
|---|---|---|---|---|
| | | | Power (W) | Irradiance (power density), W/cm² |
| | Wavelength | | or | or |
| Laser Type | (nm) | Mode(s)¹ | Energy (J) | Fluence (energy density), J/cm² |
| Carbon dioxide | 10,600 | CW | 100 W | 15,000 W/cm² |
| | | Super-pulsed | >2 KW (peak) | 10,000 W/cm² Avg. |
| Holmium | 2,100 | Pulsed | 15 W Avg. | 200 W/cm² Avg. |
| Nd:YAG | 1,064 | CW & Pulsed | 100 W Avg. | 15,000 W/cm² non-contact |
| | | | | 3,000 W/cm² contact |
| Diode | 800-980 | CW | 25 W | 3,000 W/cm² |
| 2^{nd} harmonic | 532 | Pulsed | 20 W Avg. | 2,000 W/cm² Avg. |
| Nd:YAG | | | | |
| Argon | 488/514 | CW | 20 W | 2,000 W/cm² |
| Excimer | | | | |
| ArF | 190 | Pulsed | 600 mJ/pulse | 75 J/cm² |
| XeCl | 308 | Pulsed | 300 mJ/pulse | 40 J/cm² |
| Er:YAG | 2,940 | Pulsed | 700 mJ/pulse-4,000 mJ/ | <1 J/cm² to >25 J/cm² |
| | | | pulse | 15 J/cm² Avg. |

¹Continuous Wave (CW) mode includes gating the laser to a predetermined duration (typically in the range of 0.1-2 sec) and frequency.
Pulse Mode is typically in the range of 0.05 ms-10 ms). This mode also includes a special case of “super-pulsed” that is generally defined for high peak power, repetitive pulses from a carbon dioxide laser. Pulsed mode can also include Q-switched (0.1 ns-100 ns) not described herein.

If the determination is made that the fingerprint of target tissue 130 is normal, the procedure can proceed differently depending on the required or desired result (S6). Probe 70 of medical device 10 can move to the next anatomical location (S7). The new target tissue can be directly adjacent to target tissue 130, or can be any other tissue site requiring attention. At the new target tissue, diagnosis will be performed in the same manner as discusses above, beginning with step S1.

It may be necessary to diagnose remaining target tissue 130 after the therapeutic ablation or to diagnose subcutaneous tissue below a tissue layer. A decision can be made by the controller 40 or surgeon to deliver ablative energy (S5) to the same target tissue 130 rather than move to another anatomical location (S7). If this decision is made, probe 70 may remain on target tissue 130 and controller 40 will trigger energy source 20 to ablate the tissue even though it has a normal fingerprint. In this case, the “diagnostic” ablation can be done, for example, to diagnose the tissue lying underneath the normal tissue. This step is particularly important when diagnosing and treating at the edges of abnormal masses to ensure the entire abnormality is removed. During this procedure, for example, the fingerprints of denatured tissue are used to determine normalcy or malignancy based on tissue that has been ablated one or more times. For example, with basal cell carcinomas, the malignant tissue can be hidden by normal tissue on the surface while the malignancy is growing underneath. Some anomalies may be known to be entirely under one or more layers of normal tissue, requiring the normal tissue to be removed to access the anomalous tissue. After ablating the normal target tissue 13 0, medical device 10 can then proceed to diagnosis (SI). As used herein, “normal target tissue” can be normal tissue or denatured normal tissue. As noted, whether to move to a new target tissue site or ablate the normal tissue can be decided by the surgeon before or during treatment. It is contemplated that controller 40 can be preprogrammed with specific dimensions or with a specific sequence of the steps described above. A non-limiting example of a programmed dimension is continued diagnosis until reaching one millimeter beyond and/or below the last anomalous fingerprint. A non-limiting example of a specific sequence might be to repeat the therapeutic sequence three times after an anomalous fingerprint and before performing another diagnosis. Any combination of diagnosis and therapeutic and diagnostic ablation can be programmed in controller 40 and used by one skilled in the surgical art. It is also contemplated that the surgeon can determine the necessary sequence during treatment or over ride a programmed sequence as required. Alternatively, a triggering device within or connected to controller 40 can initiate the necessary sequence based on pre-programmed information.

Display 80, shown in FIG. 1, can be used for several functions. Non-limiting examples of uses of display 80 include viewing the fingerprints, setting the operating parameters, providing real time clinical data, such as accumulated power or energy, duration of treatment, or patient information. Further, display 80 is optional and may not be required in certain circumstances.

Probe 70 of medical device 10 can be manually driven by the surgeon during treatment. Due to the minute scale and precise nature of the diagnosis and treatment, probe 70 can also be robotically driven. For example, a robot mechanism can be driven by 40 to precisely control the location of probe 70 during the treatment process. The robot mechanism can be, for example, an articulated robotic arm. Alternatively, the robotic apparatus can be an optical scanner. These robotic devices are provided by way of example and not limitation, and other robotic apparatus known in the art can be used to control the movement of the probe.

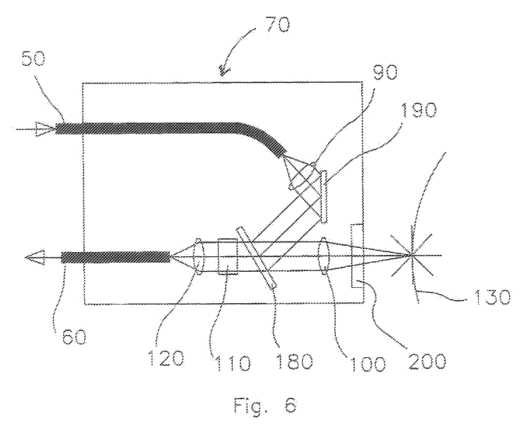

Probe 70 is not limited to the embodiment described above. Probe 70 can be configured with a specimen-engagement portion that physically contacts the target tissue to be diagnosed and/or treated. Another probe embodiment is shown in FIG. 6. Probe 170 comprises at least a portion at the distal ends of excitation/ablation conduit 50 and sensing, or collector, conduit 60. However, in this embodiment, the excitation/ablation wavelength path and the detection path are coaxially aligned to the tissue. In order to accomplish the coaxial orientation, a dichroic beam splitter 180 is used to combine the two optical paths. Any similar device known in the art can be used to function similar to the dichroic beam splitter. As in the first embodiment, lens 90 can be in the path from the distal end of excitation/ablation conduit 50. Lens 90 focuses the beam on mirror 190, which redirects the excitation/ablation energy to dichroic beam splitter 180, which directs the beam to target tissue 130. The scatter can pass from target tissue 130 through first lens 100, filter 110 and second lens 120 as in the first embodiment.

Also shown in this embodiment is protective window 200 on the distal end of probe 170. Protective window 200 prevents debris from entering probe 170 and decreasing the life of the fiber optics. Protective window 200 can be made of quartz (fused silica), sapphire or a material known by those skilled in the art with similar optical characteristics. Protective window 200 is removable and easily cleaned or replaced as desired or required. Although protective window 200 is shown in FIG. 6 in probe 170, protective window 200 can be incorporated into any probe embodiment discussed herein.

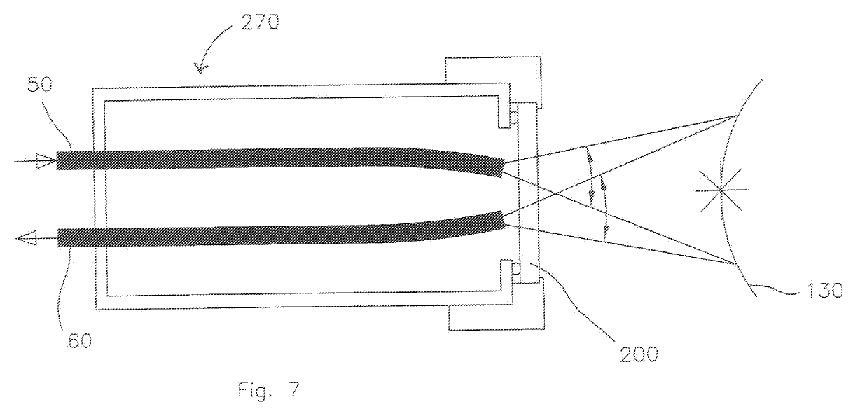

A third embodiment of a probe for use with medical device 10 is shown in FIG. 7. In this embodiment, no focusing optics in or out of the distal end of the probe 270 are used. Protective window 200, described above, is shown as easily replaceable, optionally disposable, and is incorporated into this embodiment.

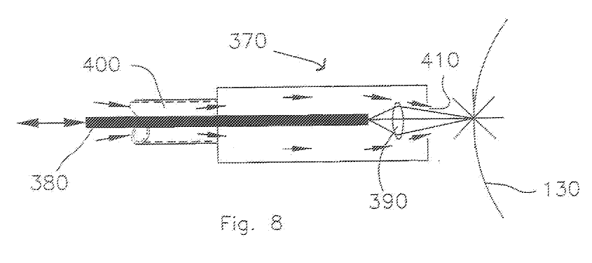

A fourth embodiment of a probe for use with medical device 10 is shown in FIG. 8. Probe 370 comprises a single conduit 380 that can deliver excitation/ablation energy to target tissue 130 and a lens 390 to focus the energy. In addition, the same conduit 380 can collect the scatter through the same probe 370 and deliver the scatter to the sensor (not shown). Probe 370 can also comprise inert gas catheter 400 that can deliver positive pressure air, nitrogen or other inert gas through hole 410 in the distal end of probe 370. The positive pressure gas blowing through inert gas catheter 400 can protect the contents of probe 370 from debris. As used herein, “debris” is anything resulting from the ablation of the tissue, such as a plume of smoke, blood, ablated tissue remains, and other bodily fluid.

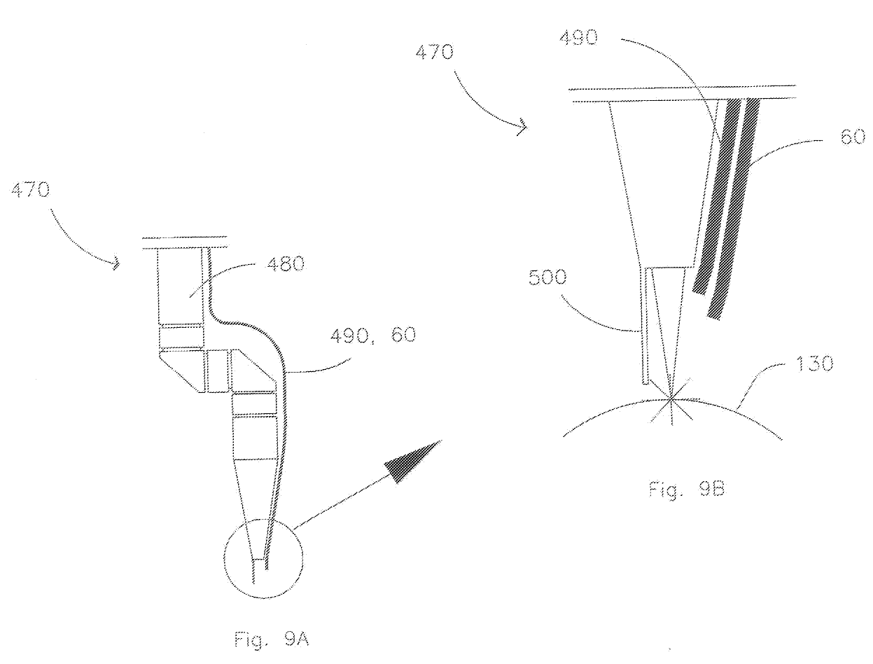

A fifth embodiment of a probe for use with medical device 10 is shown in FIGS. 9A and 9B. Probe 470 comprises hollow articulating arm 480 such as that used to carry near infrared, carbon dioxide laser emissions from energy source 20 (not shown). FIG. 9A illustrates the entire distal end of articulating arm 480. Adjacent to articulating arm 480 run excitation conduit 490 and sensing conduit 60. A diagnostic excitation beam is delivered to target tissue 130 through excitation conduit 490 from energy source 20 (not shown) or a separate energy source as desired or required. Sensing conduit 60 performs as described above. FIG. 9B is an exploded view of the very distal end of probe 470, showing aiming device 500 used to aid in directing the ablation energy from the carbon dioxide laser to target tissue 130.

It is contemplated that other useful devices may be incorporated into the probe embodiments as desired or required. For example, a vacuum removal tube can be configured to remove debris from the tissue area after ablation has occurred. The vacuum tube can transmit the debris to a chamber (not shown) attached to the distal end. The chamber can be any specimen or waste container well known and used in the art. Alternatively to or in addition to the chamber, a gas spectrometer (not shown) can be connected to vacuum tube for spectrometric analysis of the tissue debris.

A camera may be incorporated into the medical device to capture images of the procedure or target tissue. The camera can be a still camera or a video camera as desired or required.

Another example that may be incorporated into the probe embodiments is an electrosurgical conduit. The electrosurgical conduit can contain an electrosurgical device configured as a cautery or hemostatic waveguide to maintain hemostasis after the target tissue has been ablated. The electrosurgical conduit can also contain a cutting or ablating device as desired or required. The end of the electrosurgical conduit opposite the target tissue can comprise, for example, a heat source to cauterize the treated tissue with heat or a caustic source to cauterize the treated tissue with caustic. These are provided by way of example and not limitation, and other cauterization devices known in the art may be used. The electrosurgical conduit can take the place of or be used in addition to a laser used to produce ablative energy. Controller 40 can be configured to control the electrosurgical conduit alone or in addition to an energy source.

The target tissue can be any tissue to which the probe of the medical device can reach, for example, skin tissue. Any of the probes disclosed herein can be located on the end of an endoscope, laparoscope, intervaginal probe, bronchoscope, cystoscope, or any similar device known in the art to diagnose and treat internal tissue anomalies.

When used internally on the end of an endoscope, for example, any of probe embodiments discussed above can be farther equipped in the end of an endoscope or similar device. The probe embodiments discussed herein may further comprise a light carrying fiber optic with a visualization optic. The light carrying fiber optic and visualization optic allow direct visualization of the probe tip and target tissue site during diagnosis and treatment of internal tissues. Visual display of the probe tip and target tissue site can be produced on a display device well known in the art and depicted as display 80 in FIG. 1.

Also disclosed herein are methods for diagnosing and treating tissue anomalies. One such method comprises the following steps, as outlined in FIG. 3. The probe as described in one of the embodiment herein is positioned proximate a target tissue. An excitation beam is delivered from an energy source to the target tissue through the probe. An at least one predefined attribute of radiation, or scatter, reflected or emanated by the target tissue is collected with the probe and relayed to a sensor for fingerprinting. The fingerprint is analyzed against the tissue fingerprint database. The fingerprint is identified as a normal fingerprint or an anomalous fingerprint and a signal is sent from the controller to the energy source to actuate one of the following: deliver a therapeutic ablative energy from the energy source through the probe at least sufficient to ablate at least a portion of the target tissue with the anomalous fingerprints deliver a diagnostic ablative energy from the energy source through the probe at least sufficient to ablate at least a portion of the target tissue with the normal fingerprint, or position the probe relative to another target tissue to repeat the method until the diagnosing and treating process of a tissue area is complete.

Another embodiment of a method for diagnosing and treating tissue anomalies comprises positioning a probe coupled to an energy source proximate a target tissue; delivering one of an excitation energy and an ablative energy through the probe from the energy source to the target tissue depending on a signal from a controller; capturing a scatter reflected from the target tissue with the probe when excitation energy has been delivered, relaying the scatter to a spectrometer and fingerprinting the scatter's spectra against a tissue fingerprint database in the controller; and providing the signal from the controller to the energy source.

The actuation of the particular beam of energy is determined by a surgeon or pre-programmed in the controller. The method is repeated until all of the target tissue has been diagnosed and treated. To move the probe from target tissue site to target tissue site, a robotic apparatus can be used as discussed above, or the probe can be moved manually.

The probe can comprise a first conduit and a second conduit, so that the first conduit delivers the excitation and ablation energy and the second conduit senses the scatter. Alternatively, the excitation beam may be delivered through a conduit in addition to the conduit that delivers the ablation energy. The probe can further comprise an inert gas catheter as described above.

With procedures in which the probe is positioned in an endoscope or the like, a light carrying fiber optic with a visualization optic can be employed during the diagnosis and treatment for directly viewing the probe tip and the target tissue site.

The methods can further comprise storing the fingerprints received by the sensor in the database and displaying a virtual biopsy on a display device.

Advantages of the medical devices and methods disclosed herein are significant. The medical device and procedure disclosed herein can non-invasively diagnose anomalies in tissue and can contemporaneously treat any tissue positively diagnosed. By only ablating the tissue that requires it, surrounding healthy tissue is left in tact, providing a better tool for areas of tissue where cosmesis is a concern. For tissue located on the integument, subcutaneously or with a cavity of the body, the device and procedure provide real time diagnosis without having to biopsy a sample, analyze the sample, and later treat the area based on the tissue sample removed. Samples of tissue do not have to be frozen, which can decrease accuracy of diagnosis. This list is exemplary. Many more advantages can be realized by those skilled in the art.

While the invention has been described in connection with certain embodiments, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A medical device for diagnosing and treating anomalous tissue comprising:
an energy source configured to emit at least an excitation beam and a therapeutic beam;
a probe coupled to the energy source and configured to propagate the excitation and therapeutic beams, the beams capable of contact with the tissue;
a sensor coupled to the probe that detects at least one predefined attribute of radiation emanating from the tissue when the tissue is subjected to the excitation beam; and
a controller coupled to the energy source and the sensor and programmed to selectively alternatively actuate the energy source to emit the excitation beam and the therapeutic beam in response to the detection of the at least one predefined attribute by the sensor.

2. The medical device of claim 1, wherein the excitation beam is light of a wavelength suitable for exciting the tissue to emit the at least one predefined attribute of radiation.

3. The medical device of claim 1, wherein the energy source is configured to emit the therapeutic beam in at least one of a therapeutic ablation mode and a diagnostic ablation mode in response to the controller.

4. The medical device of claim 1, wherein the therapeutic beam is an emission of electromagnetic energy, heat or ultrasound.

5. The medical device of claim 1, wherein the at least one predefined attribute produces a fingerprint associated with a pathology.

6. The medical device of claim 1, wherein the sensor comprises a spectrometer configured to extract a spectrum from the radiation emanating from the tissue, and the probe comprises:
a collector arranged to collect radiation emanating from the tissue; and
a conduit coupling the collector and the spectrometer to permit the radiation to propagate from the collector to the spectrometer.

7. The medical device of claim 6, further comprising:
memory containing at least one fingerprint associated with one or more of an anomalous condition and a normal condition, wherein at least one of the controller or the sensor is configured to compare the extracted fingerprint with the at least one fingerprint contained in the memory.

8. The medical device of claim 1, wherein the energy source comprises a first emitter capable of generating the excitation beam and the second emitter capable of generating the therapeutic beam.

9. The medical device of claim 1, wherein the probe further comprises:
a distal end movable into proximity of the tissue and having a lens aperture through which the excitation beam and therapeutic beam may be emitted; and
a conduit coupling the energy source to the lens aperture and coupling the collector to the sensor, and
wherein the sensor comprises a spectrometer configured to extract a spectrum from the radiation emanating from the tissue.

10. The medical device of claim 9, wherein the conduit comprises at least one of a single fiber optic strand and a pair of fiber optic strands.

11. The medical device of claim 9, wherein the probe further comprises a dichroic beam splitter and a mirror.

12. The medical device of claim 9 further comprising memory containing at least one fingerprint associated with at least one of a pathology and normal tissue, wherein at least one of the controller or the spectrometer is configured to generate a control signal indicative of whether the extracted fingerprint matches the at least one fingerprint in the memory.

13. The medical device of claim 12, wherein the controller is responsive to the control signal to selectively actuate the energy source to emit the excitation beam and the therapeutic beam when at least one of the sensor and the controller detects the existence of the predefined attribute.

14. The medical device of claim 13, wherein the controller is responsive to the control signal to selectively actuate the energy source to emit the therapeutic beam in at least one of a therapeutic ablation mode and a diagnostic ablation mode.

15. The medical device of claim 9, further comprising a robot mechanism responsive to the controller and configured to move the probe relative to the tissue, wherein the controller is programmed to repeatedly: actuate the energy source to emit the excitation beam; in response to the control signal, actuate the energy source to emit the therapeutic beam in at least one of a therapeutic ablation mode and a diagnostic ablation mode; actuate the robot mechanism to move the probe.

16. The medical device of claim 1, wherein the probe further comprises an inert gas catheter configured to blow inert gas through a hole at a distal end of the probe coaxially with the therapeutic beam.

17. The medical device of claim 1, wherein the probe further comprises a lens aperture through which the excitation beam and therapeutic beam are projected.

18. The medical device of claim 1, wherein the probe is located in at least one of an endoscope, an intervaginal probe, a laparoscope, a bronchoscope, a cystoscope, and an instrument configured for insertion into the tissue.

19. A medical device for diagnosing and treating anomalous tissue comprising:
a probe;
a spectrometer coupled to the probe;.
a database of tissue fingerprints;
a controller coupled to the probe and the spectrometer;
a first energy source coupled to the probe and configured to emit one or more of a diagnostic excitation, a therapeutic ablation and a diagnostic ablation as directed by the controller or a user on a target tissue,
wherein the first energy source delivers excitation energy through the probe to the tissue during the diagnostic excitation and the spectrometer receives a scatter from the diagnostic excitation and identifies the scatter against the database, the controller receiving a signal from at least one of the spectrometer and controller of normal or abnormal,
wherein the first energy source delivers ablative energy through the probe to an anomalous target tissue during the therapeutic ablation depending on the signal, and
wherein the first energy source delivers ablative energy through the probe to normal target tissue during the diagnostic ablation depending on the signal.

20. The medical device of claim 19 further comprising a second energy source, wherein the first energy source is configured to deliver the excitation energy and the second energy source is configured to deliver the ablative energy.

21. The medical device of claim 19 further comprising at least one robotic apparatus configured to control a movement of the probe along a tissue area to be examined, wherein the robotic apparatus is responsive to one or both of the controller and the user.

22. The medical device of claim 19, wherein the probe further comprises a light delivery fiber optic and a visualizing fiber optic.

23. The medical device of claim 19, wherein the database is further configured to store the identified scatter as fingerprints received by the spectrometer for at least one of populating the database and displaying a virtual biopsy.

24. The medical device of claim 19, wherein the scatter is Raman scatter.

25. The medical device of claim 19, wherein the database of tissue fingerprints comprises fingerprints of one or more of normal tissue, malignant tissue, denatured normal tissue and denatured malignant tissue.

26. The medical device of claim 19, wherein the energy source is an electromagnetic energy source.

27. The medical device of claim 26, wherein the electromagnetic energy source provides more than one form of electromagnetic energy.

28. The medical device of claim 19, wherein the probe is a plasma scalpel.

29. The medical device of claim 26, wherein the electromagnetic energy source is both a laser source and an electrosurgical generator.

30. The medical device of claim 19, wherein the probe is an electrosurgical electrode.

31. A medical device for diagnosing and treating tissue comprising:
a first laser;
a Raman spectrometer;
a controller;
a database of tissue fingerprints coupled to the spectrometer and controller; and
a probe coupled to the controller and comprising an excitation/ablation fiber optic and a sensing fiber optic, wherein the first laser is configured to:
deliver excitation light through the excitation/ablation fiber to the target tissue, such that the target tissue emits Raman scatter collected in the sensing fiber optic and delivers the Raman scatter to the Raman spectrometer for comparison to the database;
deliver ablative laser energy through the excitation/ablation fiber optic to an anomalous target tissue, and
deliver laser energy through the excitation/ablation fiber optic to a normal target tissue.

32. The medical device of claim 31, wherein the probe further comprises a protective window on a distal end of the probe.

33. The medical device of claim 3 1, wherein the probe further comprises a lens between a distal end of the excitation/ablation fiber optic and a distal end of the probe, and a first tens, a filter, and a second lens between a distal end of the sensing fiber optic and the distal end of the probe.

34. The medical device of claim 31, wherein the probe further comprises a lens and a mirror between a distal end of the excitation/ablation fiber optic and a distal end of the probe, and a first lens, a dichroic beam splitter, a filter, and a second lens between a distal end of the sensing fiber optic and the distal end of the probe.

35. The medical device of claim 31 further comprising a second laser, wherein the first laser is configured to deliver the excitation light to the excitation/ablation fiber optic and the second laser is configured to deliver the ablative energy to the excitation/ablation fiber optic.

36. The medical device of claim 31, further comprising at least one robotic apparatus configured to control a movement of the probe along a tissue area to be examined, wherein the robotic apparatus is driven by a controller or a user.

37. A method of diagnosing and treating anomalous tissue comprising:
positioning a probe coupled to an energy source proximate a target tissue;
delivering one of an excitation energy and an ablative energy through the probe from the energy source to the target tissue depending on a signal from a controller;
capturing a scatter reflected from the target tissue with the probe when excitation energy has been delivered, relaying the scatter to a spectrometer and fingerprinting the scatter's spectra against a tissue fingerprint database in the controller; and
providing the signal from the controller to the energy source.
